(19) United States
(12) Patent Application Publication
(10) Pub. No.: US 2021/0115483 A1
(43) Pub. Date: Apr. 22, 2021

(54) Transparent Cellulose-Based Materials and Methods of Making the Same

(71) Applicant: Worcester Polytechnic Institute, Worcester, MA (US)

(72) Inventors: Jeannine M. Coburn, Worcester, MA (US); Elizabeth M. van Zyl, Worcester, MA (US); Eric M. Young, Worcester, MA (US)

(21) Appl. No.: 17/073,088

(22) Filed: Oct. 16, 2020

Related U.S. Application Data
(60) Provisional application No. 62/916,649, filed on Oct. 17, 2019.

(51) Int. Cl. C12P 19/04; A61L 15/12; A61L 15/14; A61L 27/20; A61L 27/50

(52) U.S. Cl. CPC C12P 19/04; A61L 15/12; A61L 27/50; A61L 27/20; A61L 15/14

(57) ABSTRACT

Provided herein are transparent bacterial-derived cellulose-based materials having a light transmission of at least 50% of light energy having a wavelength between 400 nm and 600 nm. In some embodiments, such materials can be prepared by a method comprising culturing cellulose-producing bacteria in a media comprising a non-glucose carbon source and collecting cellulose-based material produced by the cellulose-producing bacteria, wherein the cellulose-based material has a light transmission of at least 50% of light energy having a wavelength between 400 nm and 600 nm.

### RELATED APPLICATIONS

This application claims the benefit of and the priority to U.S. Provisional Patent Application Ser. No. 62/916,649, filed Oct. 17, 2019, the entire disclosure of which is herein incorporated by reference in its entirety.

### FIELD

The present disclosure relates to transparent bacterial-derived cellulose-based materials, and methods and kits for making and using the same.

### BACKGROUND

Bacterial-derived cellulose has become a prominent material for biomedical research applications due to its ease of fabrication, biocompatibility, high yield strength, and water retention properties. Optical transparency, however, has not been consistently observed in pure bacterial-derived cellulose and has predominantly been achieved through the addition of composite materials or coatings. Increased transparency of the bacterial-derived cellulose would allow for a broader range of biomaterial applications, such as wound dressing, corneal tissue engineering, and biosensing. Existing chemical modification and composite formation techniques for cellulosic materials have been employed to formulate water soluble or highly swollen cellulose that then gives rise to optically clear properties. Past approaches to produce optically clear cellulose include the use of polyurethane and poly(2-hydroxyethyl methacrylate) and composite materials or chemical conversion of cellulose to hydroxymethylcellulose. New approaches that minimize steps to synthesize optically transparent films and reduce or eliminate costly or toxic chemical reactions are urgently needed.

### SUMMARY

As described below, the present disclosure features transparent bacterial-derived cellulose-based materials, for example, pellicles, films, and hydrogels.

In some aspects of the present disclosure, a method is provided for preparing a transparent bacterial-derived cellulose-based material that comprises culturing cellulose-producing bacteria in a media comprising a non-glucose carbon source and collecting cellulose-based material produced by the cellulose-producing bacteria, wherein the cellulose-based material has a light transmission of at least 50% of light energy having a wavelength between 400 nm and 600 nm. In some embodiments, the media further comprises glucose. In some embodiments, the non-glucose carbon source comprises one or more of a pentose sugar, a hexose sugar, a sugar alcohol, or a disaccharide. In some embodiments, the pentose sugar is selected from the group consisting of xylose and arabinose. In some embodiments, the hexose sugar is selected from the group consisting of mannose, galactose, fructose, glucosamine hydrochloride, and N-acetylglucosamine. In some embodiments, the sugar alcohol is selected from the group consisting of arabitol and mannitol. In some embodiments, the disaccharide is sucrose. In some embodiments, the non-glucose carbon source comprises one or more of mannose, galactose, xylose, mannitol, sucrose, glucosamine hydrochloride, N-acetylglucosamine, fructose, arabinose, and arabitol. In some embodiments, the non-glucose carbon source comprises one or more of arabitol and xylose. In some embodiments, the cellulose-producing bacteria comprises bacteria from Acetobacter genus. In some embodiments, the cellulose-producing bacteria comprises one or more of DS-12, Gluconacetobacter hansenii, or Gluconacetobacter xylinus bacterial species. In some embodiments, the bacterial-derived cellulose-based material has a light transmission of at least 75% of light energy having a wavelength between 400 nm and 600 nm. In some embodiments, the oxygen tension is reduced relative to ambient. In some embodiments, the non-glucose carbon source is selected to produce increased cyclic di-GMP levels in the cellulose-producing bacteria. In some embodiments, the cellulose-producing bacteria is cultured without glucose.

In some aspects of the present disclosure, a material is provided for biomedical application comprising bacterial-derived cellulose-based material having a light transmission of at least 50% of light energy having a wavelength between 400 nm and 600 nm.

In some aspects of the present disclosure, a kit is provided for preparing a transparent bacterial-derived cellulose-based material comprising cellulose-producing bacteria, and one or more non-glucose carbon sources, wherein culturing the cellulose-producing bacteria in the presence of the one or more non-glucose carbon source produces cellulose-based material having a light transmission of at least 50% of light energy having a wavelength between 400 nm and 600 nm. In some embodiments, the cellulose-producing bacteria comprises bacteria from Acetobacter genus. In some embodiments, the cellulose-producing bacteria comprises one or more of DS-12, Gluconacetobacter hansenii, or Gluconacetobacter xylinus bacterial species. In some embodiments, the non-glucose carbon source comprises one or more of mannose, galactose, xylose, mannitol, sucrose, glucosamine hydrochloride, N-acetylglucosamine, fructose, arabinose, and arabitol.

### BRIEF DESCRIPTION OF THE DRAWINGS

The present disclosure will be described in the detailed description which follows, in reference to the noted plurality of drawings by way of non-limiting examples of exemplary embodiments, in which like reference numerals represent similar parts throughout the several views of the drawings, and wherein:

FIG. 1A and FIG. 1B comprise images of cellulose pellicles and aggregates produced in either static (FIG. 1A) or agitated (FIG. 1B) culture, respectively. Undisturbed static culturing was performed for 7 days. Agitated culturing was performed for 7 days at 200 rpm.

FIG. 1C and FIG. 1D are images of pellicles according to some embodiments of the disclosure. FIG. 1C displays a cellulose-based material of the present disclosure having a modular shape. FIG. 1D displays the attachment of fibroblasts to the cellulose pellicle, which indicates the biocompatibility of the material.

FIG. 2 is an illustration showing cellulose production in a bacterial cell and a resulting pellicle comprising nanofibril bundles visualized via scanning electron microscopy.

FIG. 3 is a genomic tree providing the results of profiling performed on the DS-12 strain bacteria, showing it to be a close relative to Komagataeibacter xylinus.

FIGS. 4-7 illustrate various metabolic pathways for producing transparent cellulose based materials from alternative carbon sources.

FIG. 8 is a flow chart of a representative process for the preparation of cellulose-producing based materials according to the present disclosure.

FIG. 9 is a flow chart for the pellicle purification process according to some embodiments of the disclosure.

FIG. 10A is a flow chart for depicting the measuring of growth kinetics and cellulose yields for cellulose-producing bacteria, according to some embodiments of the disclosure.

FIG. 10B is a graph illustrating the measurement of optical density versus time to generate a cell population growth kinetics curve of maximum growth rate (μmax), for example, for glucose-derived cellulose and arabitol-derived cellulose, according to some embodiments of the disclosure.

FIG. 11A is a graph illustrating maximum growth rate of cultures grown in media comprising different carbon sources according to some embodiments of the disclosure.

FIG. 11B is a graph illustrating the cellulose yield % for cultures grown in media comprising different carbon sources according to some embodiments of the disclosure.

FIG. 12 is an image the increased transparency of cellulose-based materials produced with alternative carbon sources (mannose, galactose, xylose) as compared to glucose in three cellulose-producing strains (DS-12, G. xylinus, and G. hansenii) according to some embodiments of the present disclosure

FIGS. 13A-13B illustrate a representative determination of % transmittance according to some embodiments of the disclosure by performing UV-Vis determinations of a light source through a sample, for example, pellicles derived from glucose and arabitol.

FIGS. 14A-14J illustrate representative determinations of % transmittance according to some embodiments of the disclosure by performing UV-Vis determination of a light source through samples produced by DS-12.

FIGS. 15A-15I illustrate representative determinations of % transmittance according to some embodiments of the disclosure by performing UV-Vis determination of a light source through samples produced by G. xylinus.

FIGS. 16A-16J illustrates representative determinations of % transmittance according to some embodiments of the disclosure by performing UV-Vis determination of a light source through samples produced by G. hansenii.

FIG. 17A and FIG. 17B are graphs illustrating differences in transmittance of pellicles derived from cultures grown on alternative carbon sources (FIG. 17A) and cultures grown in alternative carbon sources augmented with glucose (FIG. 17B), according to some embodiments of the disclosure.

FIG. 18A and FIG. 18B are graphs showing transmittance percentage (FIG. 18A) and yield percentage (FIG. 18B) for pellicles produced by cultures grown in media comprising 100% arabitol, 75% arabitol/25% glucose, 50% arabitol/50% glucose, and 100% glucose, respectively, according to some embodiments of the disclosure.

FIG. 19 is a graph showing transmittance percentage for pellicles produced by cultures grown in media comprising 100% glucose, 50% glucose/50% arabitol, and 100% arabitol, according to some embodiments of the disclosure.

FIG. 20 is a graph and image showing transmittance percentage of pellicles produced in ambient and low oxygen cultures, according to some embodiments of the disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure provides transparent bacterial-derived cellulose-based materials. In some embodiments, such materials can be derived from one or more cellulose-producing bacteria provided with one or more non-glucose carbon sources, with or without glucose.

The methods described herein allow for the formation of naturally synthesized, optically clear materials composed of pure cellulose, or combinations of cellulose and other polysaccharides. In some embodiments, the term “cellulose” refers to a polymer comprising β(1→4) linked D-glucose monomers. Cellulose is primarily derived from plant origin and is the structural component of plant cell walls and fibers of cotton, for example. Cellulose can also be bacterial-derived, and such cellulose may require minimal processing. A representative structure of cellulose is as follows:

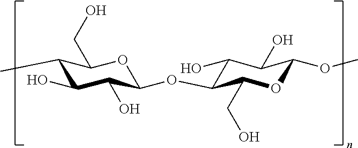

Transparent cellulose-based hydrogels have been described previously; however, fabricating these hydrogels required cross-linking or other chemical modifications. For example, transparent hydrogels have been reported that were fabricated by cross-linking 2-hydroxyethyl methacrylate (PHEMA) with bacterial-derived cellulose (see U.S. Patent Application Publication No. 20130011385). Importantly, the transparent cellulose-based materials of the present disclosure do not require additional chemical processing beyond purification (i.e., the removal of microbial components) to achieve optically transparency. In some embodiments, purification can be accomplished using methods well known in the art.

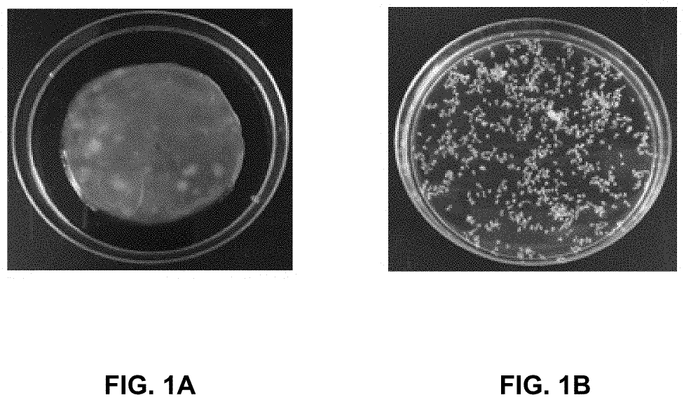

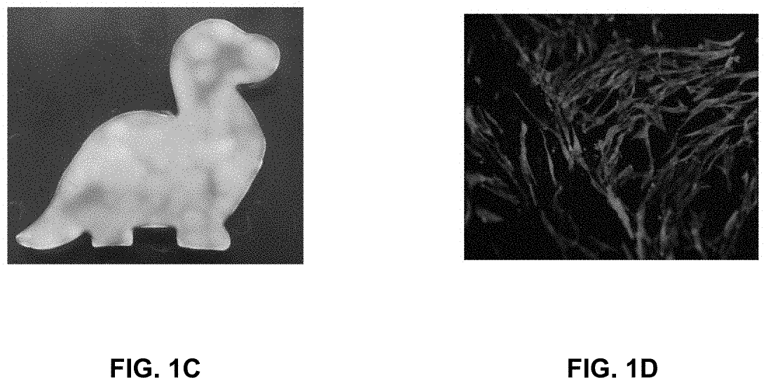

In reference to FIGS. 1A-1D, several embodiments of the transparent cellulose-based materials of the present disclosure are shown. For example, FIG. 1A displays the reticular pellicle that is produced in static bacterial culture, while FIG. 1B displays the small cellulose aggregates that form in agitated bacterial culture. FIG. 1C displays a cellulose-based material of the present disclosure having a modular shape. In some embodiments, the cellulose-based material can form in the shape of the container, demonstrating the ability to take on any desired shape. FIG. 1D displays the attachment of fibroblasts to the cellulose pellicle, which indicates the biocompatibility of the material. Cellulose pellicles were seeded with Human Foreskin Fibroblasts (HFF cells) and cultured for 5 days. Samples were then stained with Hoechst and phalloidin, to identify the cellular nuclei and actin filaments of the HFF cells. Staining was used to visualize cellular attachment to the cellulose material.

The cellulose-based materials of the present disclosure can be presented in various forms. For example, the cellulose-based materials can be presented in a form of a pellicle, fiber, membrane, film, block or hydrogel. For example, the term “pellicle” can refer to a thin skin or membrane produced by cellulose-producing bacteria. In some embodiments, the pellicle can be used to manufacture a film (dried) or hydrogel (hydrated).

The properties of the bacterial-derived transparent cellulose-based materials of the present disclosure can be particularly well suited for the use in various biomedical application where transparent materials are commonly used. In general, the bacterial-derived transparent cellulose-based materials can include one or more properties selected from: high crystallinity; high degree of polymerization; biocompatibility; high flexibility; high tensile strength; high water holding capacity; have modular shape. Transparent bacterial-derived cellulose-based materials can be used to manufacture inexpensive materials for biomedical applications. For example, transparent films made from the bacterial-derived cellulose can be used for wound dressing visualization, biosensing, and corneal tissue engineering. The modular optical clarity allows the bacterial cellulose of the present disclosure to be used for tissue engineering applications where transparent films are required (e.g., corneal tissue engineering and biosensing). Optically transparent materials can be used for wound dressings, which allow for visualization of healing without the need to disrupt the wound site.

The bacterial-derived cellulose-based materials of the present disclosure are transparent or optically clear. Transparent bacterial-derived cellulose-based materials, for example, pellicles, films, and hydrogels are described by their ability to allow light to pass through as determined by ultraviolet-visible (UV-Vis) light spectroscopy.

In some embodiments, light transmission through a material in the visible light region between 400 nm and 700 nm can be used to determine transparency or increased transparency as compared to materials synthesized under standard conditions (for example, glucose-only carbon source as will be described below).

The cellulose based materials of the present are transparent or optically clear. The term “transparent” or “optically clear” is meant that these materials allow a transmission of light energy. For example, the term “transmittance” may refer to the light passing through a sample, while the term “transparency” can refers to the property of the material. In some embodiments, the value of light transmission through the material (or transmittance) is above 50% across the visible light spectrum. In some embodiments, the presently disclosed materials have the value of light transmission above 75% or between 75% and 100%. In some embodiments, the presently disclosed materials have the value of light transmission above 80% or between 80% and 100%. In some embodiments, the presently disclosed materials have the value of light transmission above 85% or between 85% and 100%. In some embodiments, the presently disclosed materials have the value of light transmission above 90% or between 95% and 100%. In various embodiments, the presently disclosed materials have the value of light transmission between 75% and 95%, 75% and 90%, 75% and 85%; 75% and 80%, 80% and 95%, 80% and 90%, 80% and 85%; 85% and 95%, 85% and 90%; 90% and 100% or 95% and 100%. In some embodiments, the transparency of the presently disclosed material is described in terms of % improvement over a standard cellulose material. Such improvement can be, for example, between 25-50% improvement, 50-100% improvement, 100-200% improvement, 200-300% improvement, or greater than 300% improvement. In some embodiments, the value of light transmission is measured with light energy having a wavelength between about 400 nm and about 600 nm.

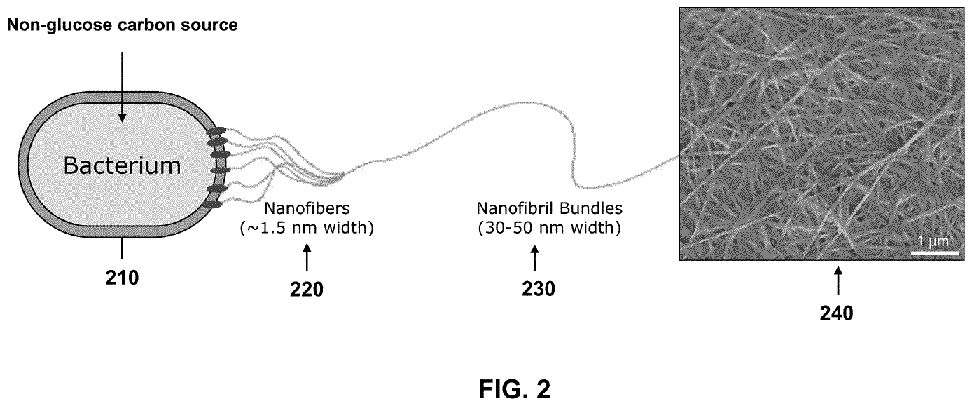

In reference to FIG. 2, to derive the transparent cellulose-based materials of the present disclosure, cellulose-producing bacteria 210 can be cultured in the presence of one or more non-glucose carbon sources. In some embodiments, the culture media used for culturing the cellulose producing bacteria according to the present disclosure may include the one or more non-glucose carbon sources prior to the culturing step. Additionally or alternatively, one or more non-glucose source can be added or supplemented during the culturing step. In some embodiments, the culturing steps may be under conditions commonly used in the art and non-limiting embodiments of the culturing conditions are described in the examples below. In response, the bacteria in the culture with the one or more non-glucose carbon source can produce nanofibers 220 that can be about 1.5 nm in width. Next, the nanofibers 220 can combine into nanofibril bundles 230 having a width of about 30 to 50 nm in width. In some embodiments, the transparent cellulose-based material (for example, pellicle) 240 is formed from the nanofibril bundles 230. In some embodiments, the transparent cellulose-based material 240 can be collected and purified for further processing.

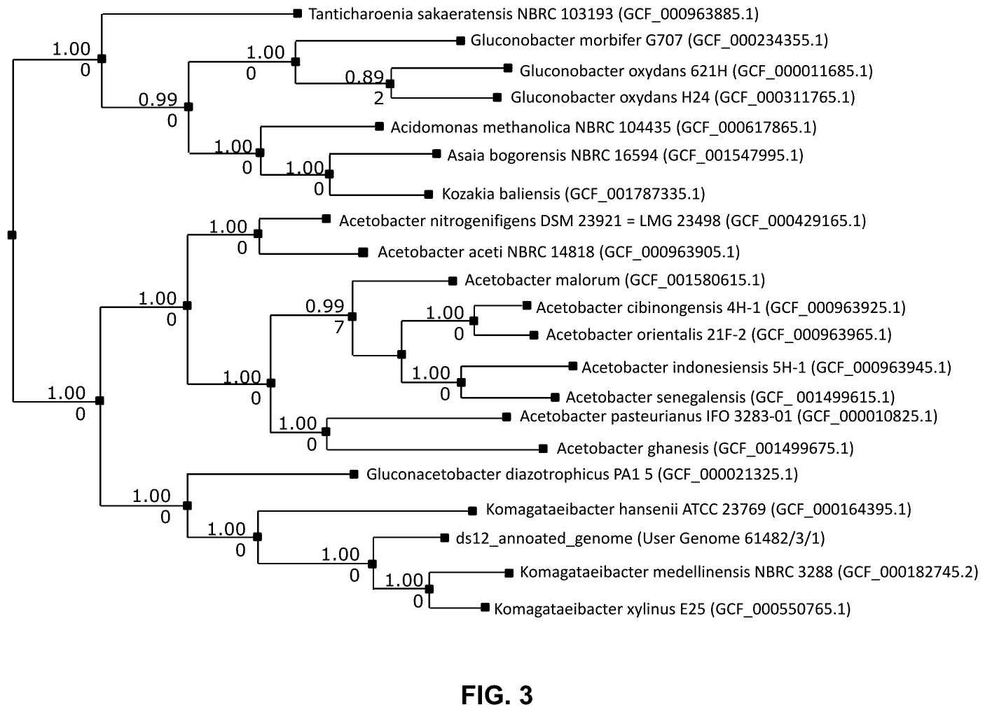

The transparent cellulose-based materials of the present disclosure can be produced by various cellulose-producing bacterial species. In some embodiments, the cellulose-producing bacteria is from an Acetobacter or Komagataeibacter genus. One or more cellulose producing strains of bacteria from Acetobacter or Komagataeibacter genus can be used. For example, in some embodiments, the cellulose-producing bacteria may be one or more of an isolated strain labeled as DS-12 (FIG. 3), Gluconacetobacter hansenii NQ5 (G. hansenii), or Gluconacetobacter xylinus (G. xylinus) (also known as Komagataeibacter xylinus and Acetobacter xylinum).

In reference to FIG. 3, the phylogenetic tree represents evolutionary relationships of the isolated DS-12 strain showing that the DS-12 strain is a unique strain in a family of cellulose-producing bacteria. The DS-12 strain is a close relative of the Komagataeibacter xylinus E25 strain and the Komagataeibacter medellinensis NBRC 3288 strain evidenced by their most common branching point. The strain naming of Komagataeibacter xylinus and Komagataeibacter medellinensis are the same species and can also be referred to as Gluconacetobacter xylinus. These two strains and the DS-12 strain are on the same branch of the phylogenetic tree and have a common branching point with Komagataeibacter hansenii ATCC23769.

In some embodiments, the transparent cellulose-based material disclosed herein are achieved by altering the carbon source used in the media culture. In some embodiments, a carbon source other than glucose can be used in the media culture. Such alternative carbon sources can be referred to as a non-glucose carbon source. In some embodiments, the non-glucose carbon source is a carbon source free of glucose. In some embodiments, the alternative carbon sources may include monosaccharides or disaccharides and may be selected from sugars or sugar alcohols. In some embodiments, the alternative, or non-glucose carbon sources include, but are not limited to, mannose, mannitol, galactose, xylose, sucrose, glucosamine hydrochloride (GlcN.HCl), N-acetylglucosamine (GlcNAc), fructose, L-arabinose, D-arabitol or combinations thereof. In some embodiments, the alternative carbon sources include, but are not limited to, pentose sugars, for example, xylose or arabinose. In some embodiments, the alternative carbon sources include, but are not limited to, hexose sugars, for example, mannose, galactose, fructose, glucosamine hydrochloride (GlcN.HCl), or N-acetylglucosamine (GlcNAc). In some embodiments, the alternative carbon sources include, but are not limited to, sugar alcohols, for example, arabitol or mannitol. In some embodiments, the alternative carbon sources include, but are not limited to, disaccharide sugars, for example, sucrose. In some embodiments, the alternative carbon source can include a combination of non-glucose carbon sources. In some embodiments, only non-glucose carbon sources can be used. In some embodiments, glucose can be used in combination with one or more non-glucose carbon sources.

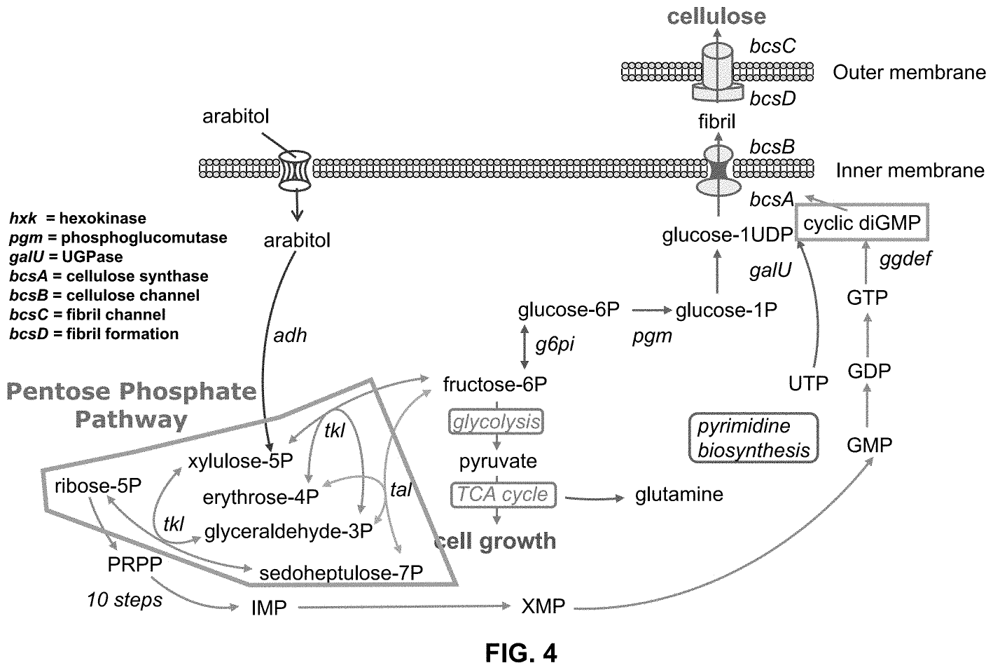

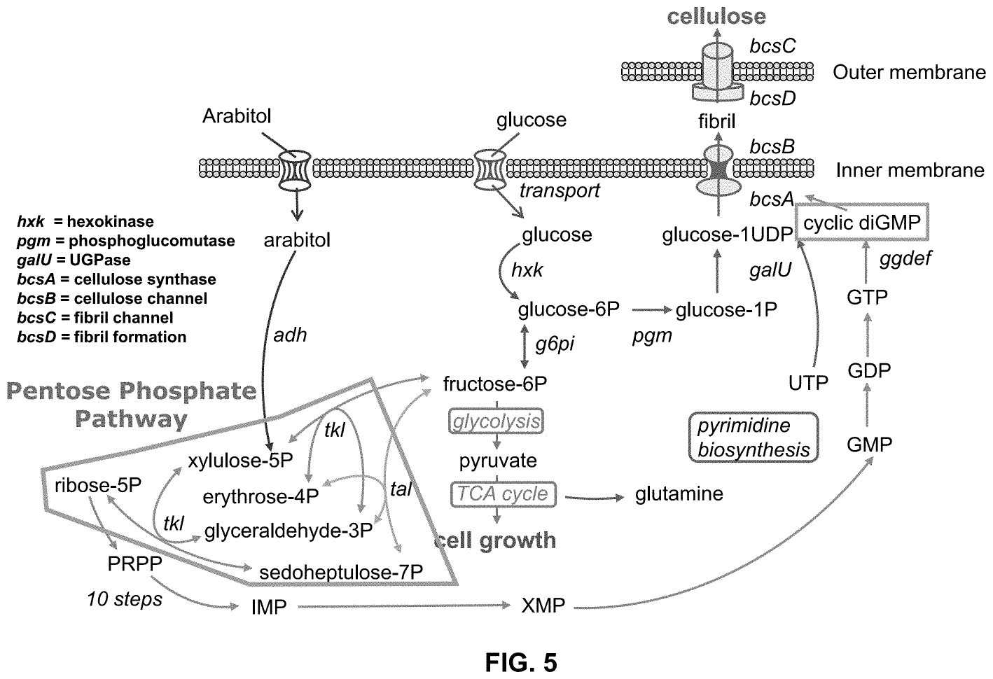

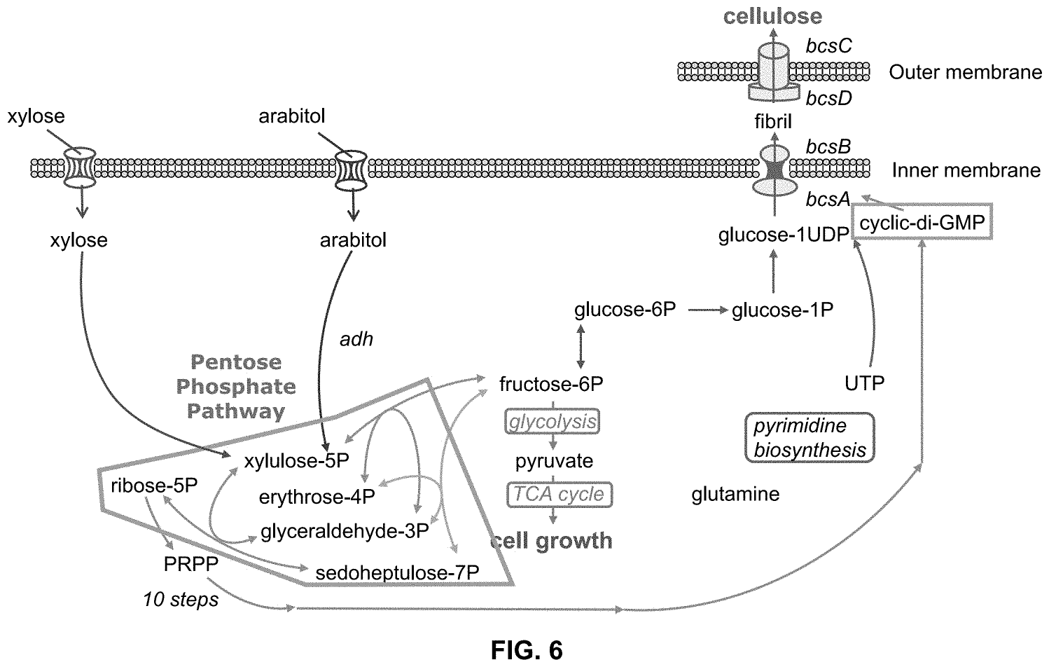

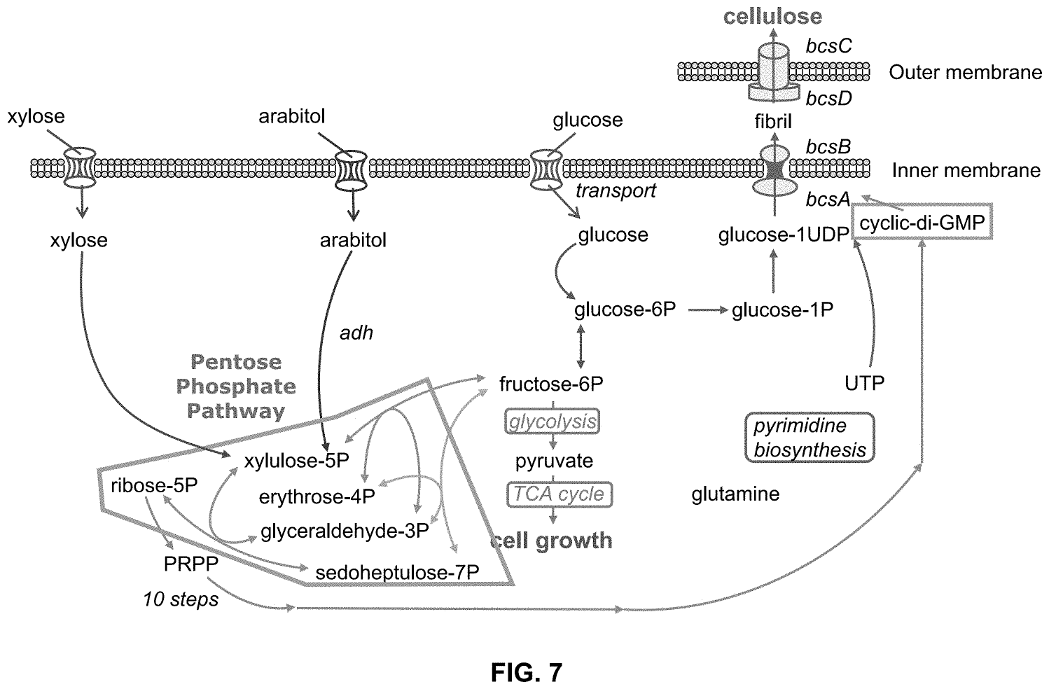

In reference to FIGS. 4-7, in some embodiments, the non-glucose carbon source is selected to activate the pentose phosphate pathway, which may lead to the production of the transparent cellulose-based materials. Further, the pentose phosphate pathway can result in increased cyclic diGMP production. In some embodiments, the increased cyclic diGMP production can lead to cellulose-based materials with increased transparency. In some embodiments, the non-glucose carbon source is selected to cause a production of cyclic diGMP. In some embodiments, the metabolic pathway for producing cellulose proceeds according the pathway shown in FIG. 4, which illustrates how the pentose phosphate pathway can convert a non-glucose carbon source (for example, arabitol) into fructose 6-phosphate, and thus result in cellulose. In some embodiments, a non-glucose carbon source (for example, arabitol) may be supplemented with glucose, so the metabolic pathway for producing cellulose proceeds according the pathway shown in FIG. 5. In some embodiments, multiple non-glucose carbon sources can be used (for example, arabitol and xylose), such that the metabolic pathway for producing cellulose proceeds according the pathway shown in FIG. 6. In some embodiments, a combination of non-glucose carbon sources may be supplemented with glucose, with the metabolic pathway for producing cellulose proceeding according the pathway shown in FIG. 7. It should be noted that while FIGS. 4-7 illustrate the metabolic pathway for the production of the transparent cellulose-based materials using arabitol and xylose, other non-glucose carbon sources can also be used as described above. In some embodiments, the alternative carbon sources can be UDP-modified and incorporated into the cellulose structure.

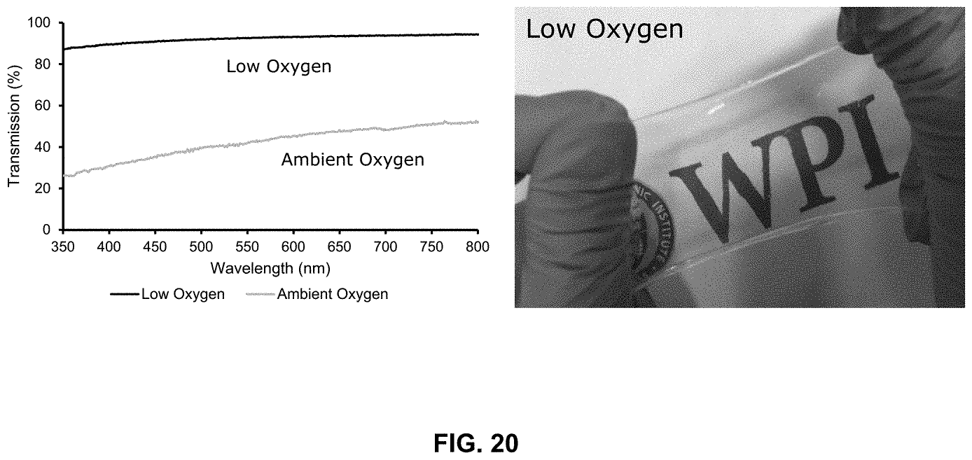

In some embodiments, the transparent cellulose-based materials disclosed herein are achieved by altering the oxygen tension (i.e., partial pressure of oxygen). In some embodiments, the transparent cellulose-based materials are produced by culturing cellulose-producing bacteria under oxygen conditions lower than ambient oxygen (e.g. within a closed container). In some embodiments, the transparent pellicles, films, and hydrogels display higher transmission % relative to those produced at ambient oxygen conditions (FIG. 20).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. For example, “or” can be understood to be either one of the elements, all of the elements, or any combination thereof. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Isolation of Cellulose-Producing Bacterial Strains

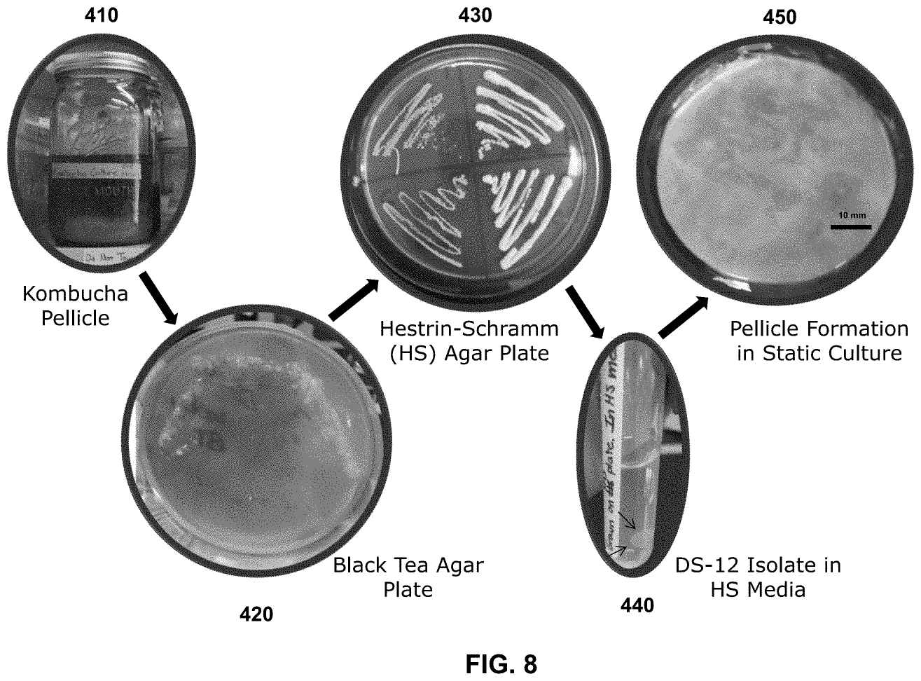

In some embodiments, the isolation of cellulose-producing bacterial strains can be accomplished by growing bacteria derived from a pellicle-producing culture on a black tea agar plate. For example, bacterial colonies growing on the plate can then be streaked onto a Hestrin-Schramm (HS) Agar plate. Colonies can then be further grown in HS media and tested for their ability to form a pellicle in static culture. An example of this process is shown in FIG. 8, which depicts the isolation of DS-12 (440), a strain closely related to Gluconacetobacter xylinus, together with the resulting transparent pellicles (450). In some embodiments, a home brewed kombucha pellicle (410) can be streaked onto a black tea agar plate (420) and allowed to grow until cellulose production is observed. In some embodiments, a single cellulose-producing colony can be restreaked on a Hestrin-Schramm media (HS) agar plate (430) and allowed to grow further. In some embodiments, single colonies from the HS plate can be used to inoculate media and allowed to grow further. In some embodiments, cellulose production ability can be confirmed. In some embodiments, the isolated strain is DS-12 (440).

### Example 2: Isolation of Cellulose-Producing Bacterial Strain DS-12

A home brewed kombucha pellicle, originally sourced from Urban Farm (Portland, Me.), was streaked onto a black tea agar plate (2 mg/mL steeped black tea was sterile filtered and supplemented with 40 mg/mL sucrose, 10% (v/v) kombucha starter, and 15 mg/mL agar) and allowed to grow at 30° C. for 4 days. Cellulose production was observed in specific colonies grown on the black tea plate. A single cellulose-producing colony was picked and restreaked on a Hestrin-Schramm media (HS) agar plate (20 mg/mL glucose, 5 mg/mL peptone, 5 mg/mL yeast extract, 1.15 mg/mL citric acid, 2.7 mg/mL disodium phosphate, 15 mg/mL agar) and allowed to grow at 30° C. for an additional 4 days. Single colonies from the HS plate were used to inoculate 5 mL of HS medium (20 mg/mL glucose, 5 mg/mL peptone, 5 mg/mL yeast extract, 1.15 mg/mL citric acid, 2.7 mg/mL disodium phosphate) media in 14 mL Falcon round cell culture tubes and allowed to grow on a rotating drum for 4 days at 30° C. Cellulose production ability was confirmed, and the isolated strain was labeled DS-12.

### Example 3: Pellicle Production

A cellulose-producing bacterial strain isolated from a home brewed kombucha pellicle DS-12 (Urban Farm Portland, Me.), Gluconacetobacter xylinus (ATCC 10245), and Gluconacetobacter hansenii (ATCC 53582) were colony picked into 5 mL of Hestrin-Schramm (HS) medium and incubated for 4 days on a shaker at 30° C. Static cultures were expanded using 10% (v/v) inoculum with the remaining being fresh medium. For experiments, fresh HS medium without glucose or mannitol was inoculated with 0.1% (v/v) static cultured bacteria. The media was supplemented to 1 mM of each carbon source under investigation (glucose, mannose, galactose, xylose, mannitol, sucrose, glucosamine hydrochloride (GlcN.HCl), N-acetylglucosamine (GlcNAc), fructose, L-arabinose, D-arabitol, and combinations thereof).

### Example 4: Pellicle Purification

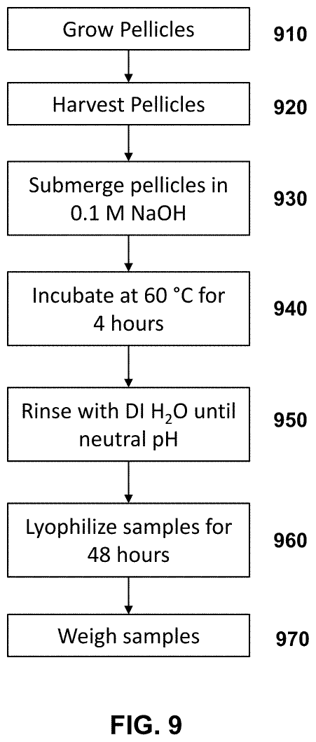

In some embodiments, cellulose purification can be by any purification method known in the art. In some embodiments, cellulose purification can be by the method described in FIG. 9. In some embodiments, after the pellicles are grown (910) and harvested (920), the pellicles can be submerged in a cleaning solution. In some embodiments, the cleaning solution can be 0.1 M NaOH (930). In some embodiments, the pellicles/cleaning solution can be incubated. In some embodiments, the pellicles/cleaning solution can be incubated at 60° C. for 4 hours (940). In some embodiments, the incubated pellicles/cleaning solution can be rinsed with deionized (DI) water until the subsequent rinse displays a neutral pH (950). In some embodiments, the pellicles can be dried. In some embodiments, the drying can be performed by lyophilization (960). In some embodiments, the pellicle yield can be determined by weighing (970). In some embodiments, the calculation for determining yield is as follows: Cellulose Yield %=(mass of pellicle/mass of carbon source added)×100.

### Example 5: Growth Kinetics

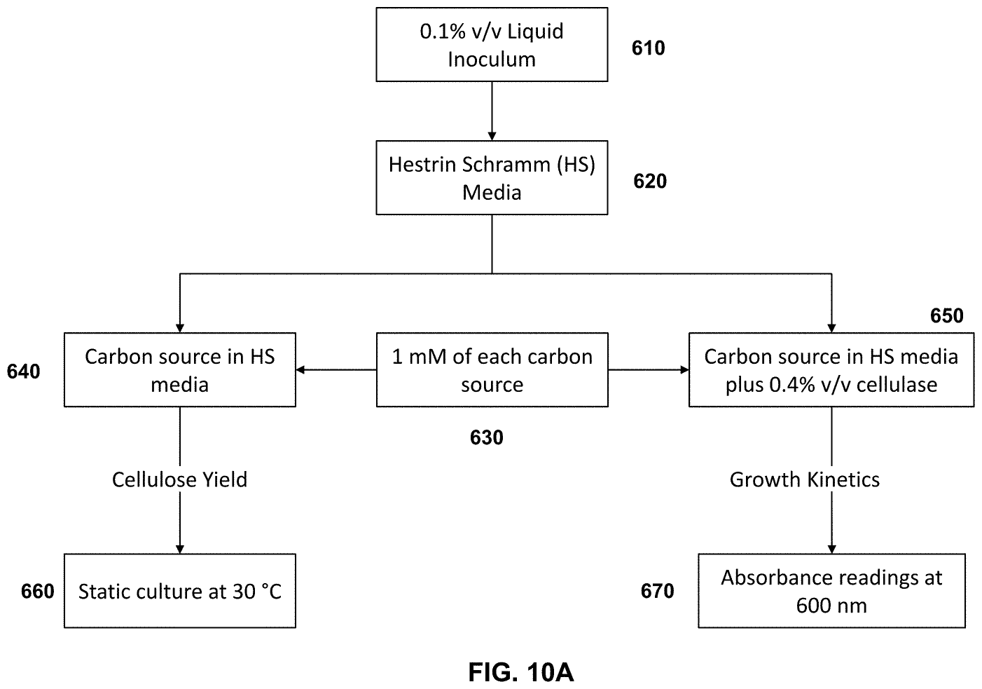

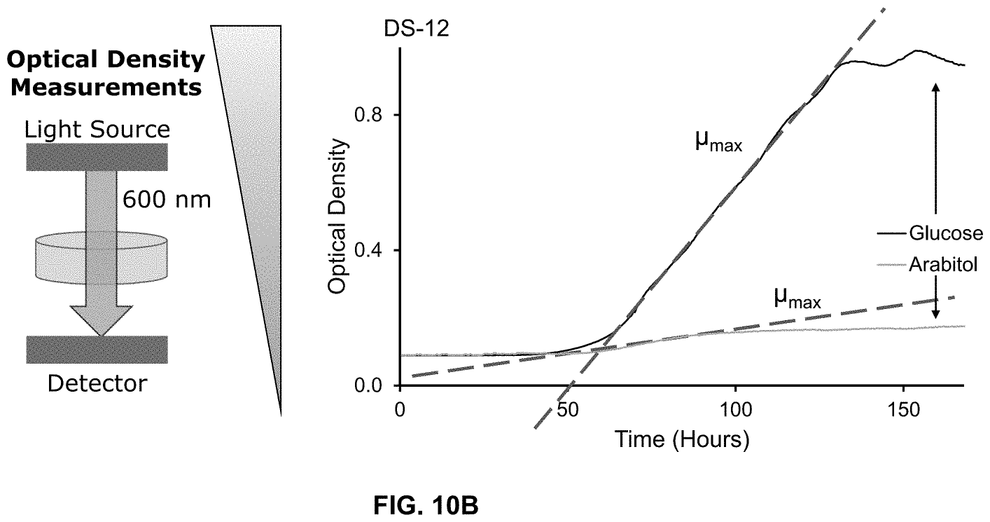

In some embodiments, the growth kinetics and cellulose yields can be characterized, for example, according to the protocol in the diagram shown in FIG. 10A. Such growth conditions can be optimized based on these types of data. Briefly, a liquid inoculum comprising a cellulose-producing bacteria (610) is grown in Hestrin-Schramm (HS) media (620). In some embodiments, 0.1% v/v liquid inoculum is fed into the HS media. In some embodiments, aliquots of the culture are transferred to individual containers (640 and 660) and fed 1 mM of a carbon source (630). In some embodiments, the combined inoculum and media can be fed the carbon source and the cellulose yield can be determined from a static culture (660). In some embodiments, the carbon source is mannose, galactose, xylose, mannitol, sucrose, glucosamine hydrochloride (GlcN.HCl), N-acetylglucosamine (GlcNAc), fructose, L-arabinose, or D-arabitol. In some embodiments, the carbon source is glucose. In some embodiments, the carbon source comprises one or more of glucose and alternative (non-glucose) carbon sources. In some embodiments, the combined inoculum and media can be treated with 0.4% v/v cellulase (650) and the growth kinetics can be determined via absorbance readings at 600 nm at various time points (670). In some embodiments, cellulose yield can be measured, for example from static cultures at 30° C. In some embodiments, the growth kinetics and/or cellulose yield from cultures that were provided alternate carbon sources can be compared to that of cultures that were provided glucose as the carbon source.

In some embodiments, a growth kinetics curve for glucose-derived cellulose can be determined. FIG. 10B is a diagram illustrating the measuring of optical density measurements of the bacteria in suspension versus time used to generate a bacteria growth kinetics curve, for example, for glucose-derived cellulose, and for arabitol-derived cellulose, according to some embodiments of the disclosure. The increase in the optical density measurement over time indicates that the bacteria are expanding in number, or growing, over time.

Optical density is used to determine bacterial population growth. These cultures were grown with cellulase preventing the cellulose pellicle from forming. As the bacterial population increased, the optical density of the culture increased. Optical density is determined using a visible light source plate reader that uses a light source at 600 nm and a light detector.

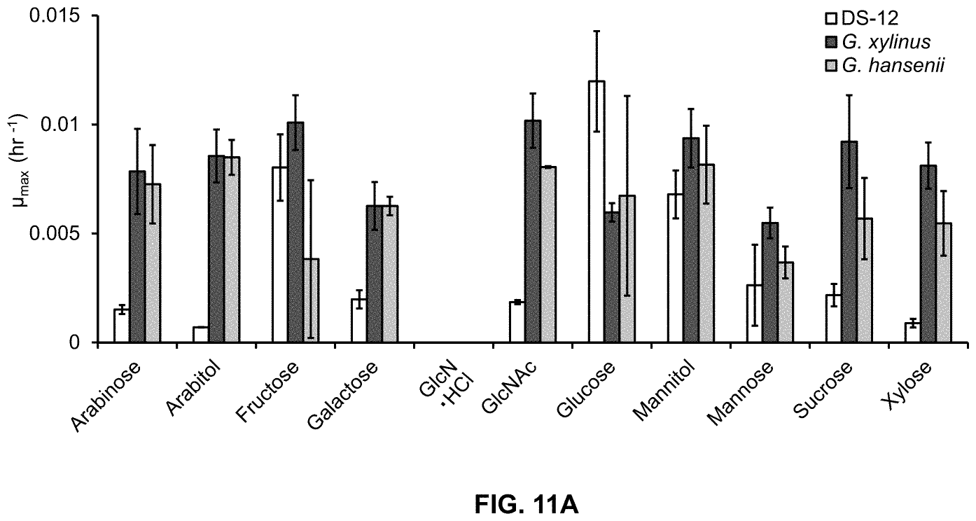

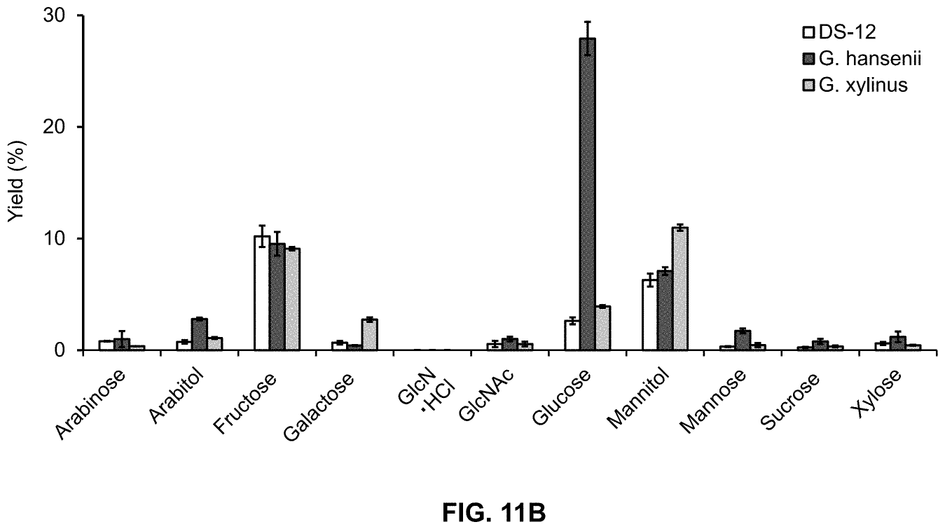

In some embodiments, the maximum growth rate for cultures grown in media comprising different carbon sources can be determined and displayed as shown in FIG. 11A. For example, FIG. 11A shows the maximum growth rate for arabinose, arabitol, fructose, galactose, GlcN(HCl), GlcNAc, glucose, mannitol, mannose, sucrose, and xylose, for DS-12, G. xylinus, and G. hansenii. In some embodiments, growth kinetic studies were performed in 96-well plates supplemented with 0.4% (v/v) cellulase from Trichoderma reesei. Absorbance measurements were taken every 30 min at 600 nm under shaken conditions (Biotek Synergy H1, Winooski, Vt.). Maximum growth rate (μₘₐₓ) values were calculated from the kinetic growth curves (see, for example, FIG. 10B). G. hansenii (ATCC strain 53582) and G. xylinus (ATCC strain 10245) showed consistent growth kinetics across all sugars tested, including increased growth rates when compared to DS-12 for arabinose, arabitol, galactose, GlcNAc, sucrose, and xylose. DS-12 showed increase growth kinetics when using glucose as a carbon source. GlcN.HCl did not support growth for any of the bacteria strains tested.

### Example 6: Cellulose Yield

In some embodiments, the yield % for cultures grown in media comprising different carbon sources can be determined and displayed as shown in FIG. 11B. For example, FIG. 11B shows % yield for arabinose, arabitol, fructose, galactose, GlcN.HCl, GlcNAc, glucose, mannitol, mannose, sucrose, and xylose, for DS-12, G. xylinus, and G. hansenii. In some embodiments, cellulose pellicles were grown in 12-well plates under static conditions at 30° C. for 7 days. Pellicles were harvested, washed in 0.1 M NaOH at 60° C. for 4 hours, and then rinsed with DI H₂O until the samples reached a neutral pH. The samples were then lyophilized using a shelf lyophilizer (Labconco, Kansas City, Mo.) for 48 hours at a temperature of −25° C. and a vacuum level of 0.210 Torr. The pellicles were weighed, and the bacterial cellulose yield was calculated as follows: % yield=[(cellulose dry weight)/(carbon source weight)]×100. DS-12 was found to support high cellulose yield when grown on fructose, mannitol, and glucose as a carbon source (FIG. 11B).

### Example 7: Transmittance Determination for Glucose and Non-Glucose Derived Pellicles

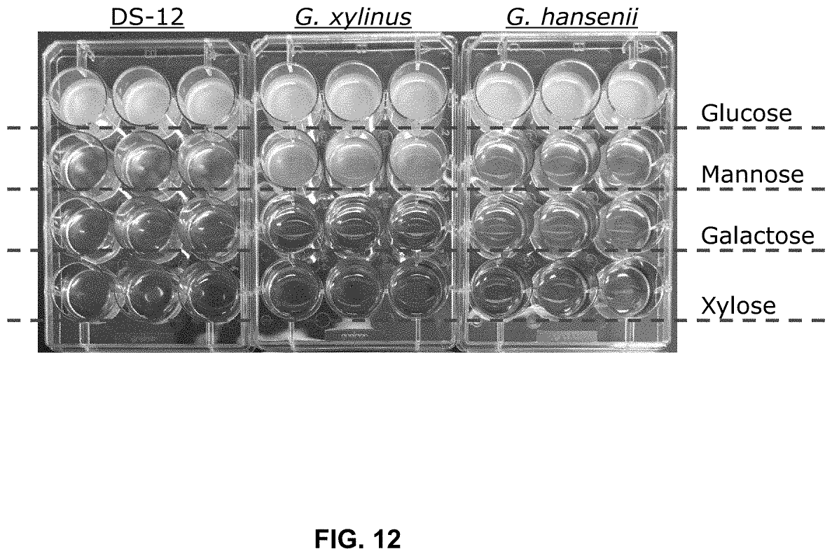

In some embodiments, alternative carbon sources (mannose, galactose, xylose) provide increased transparency as compared to glucose as demonstrated in three cellulose-producing strains (DS-12, G. xylinus, G. hansenii) as shown in FIG. 12.

In some embodiments, cellulose pellicles were grown in 12-well plates under static conditions at 30° C. for 7 days. Pellicles were harvested, washed in 0.1 M NaOH at 60° C. for 4 hours, and then rinsed with DI H₂O until the samples reached a neutral pH.

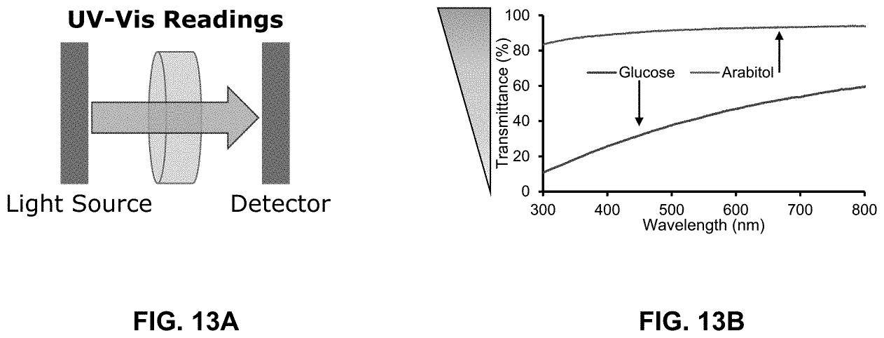

In some embodiments, pellicles were measured for overall light transmission in the visible light region using UV-Vis Spectrometry (Thermo Evolution 300 UV-Vis). For example, FIG. 13A and FIG. 13B illustrate a representative determination of % transmittance according to some embodiments of the disclosure by performing UV-Vis determinations of a light source through a sample, for example, glucose and arabitol. Samples were placed in a custom-made holder. Transmission spectral readings were done between wavelengths of 300-850 nm with a step size of 2 nm. Average transmission of light in the visible light spectrum (400-700 nm) were compared between test groups. As shown in FIG. 13B, the % transmittance of the cellulose derived using arabitol is greater than the transparency of the cellulose derived using glucose.

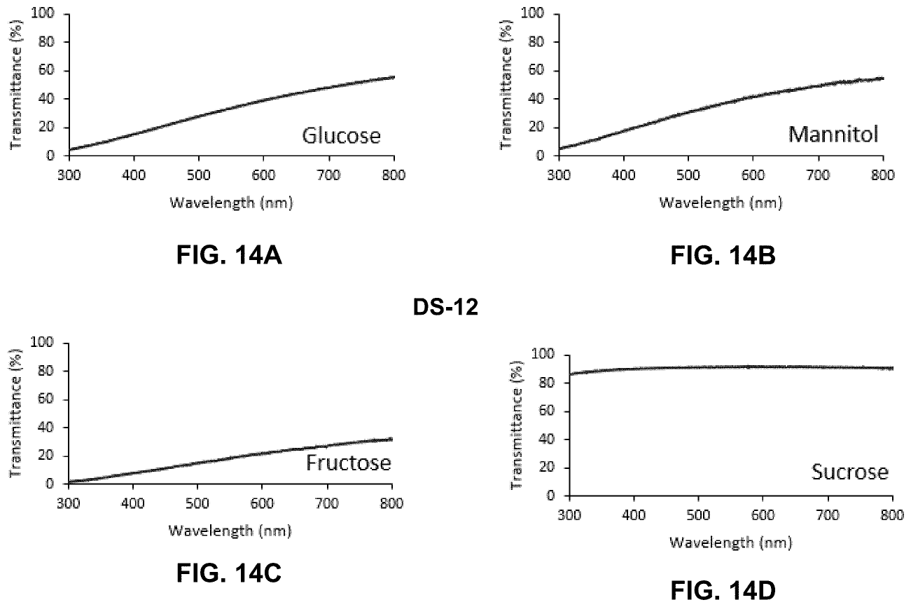

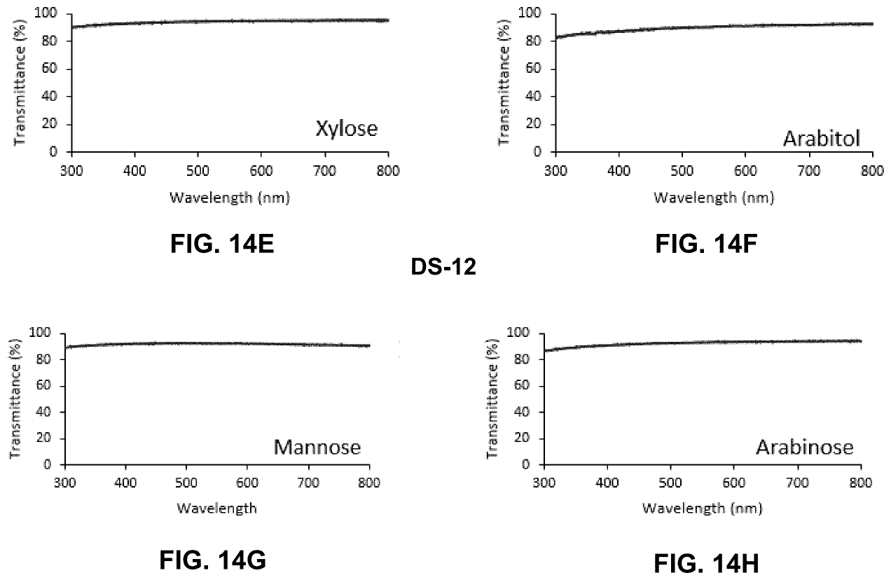

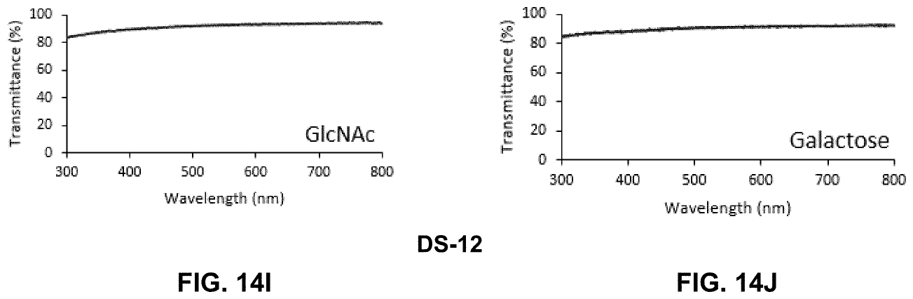

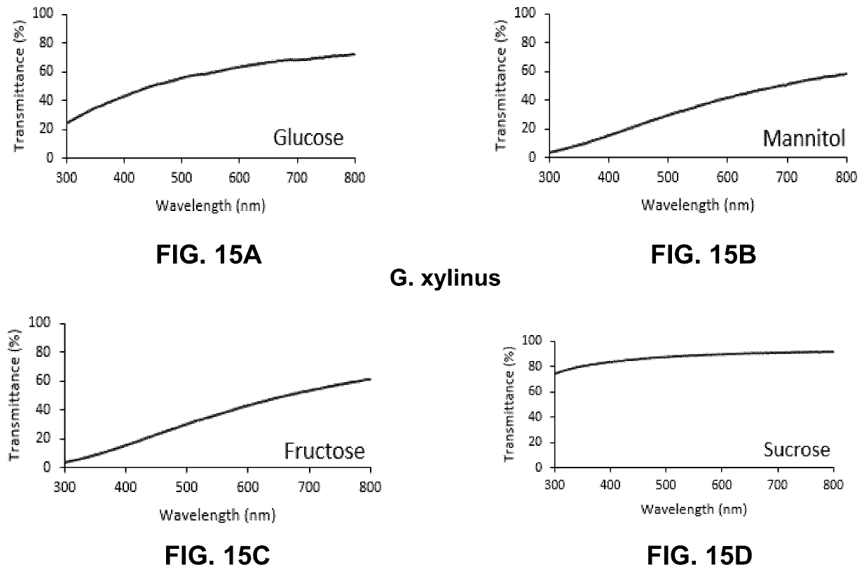

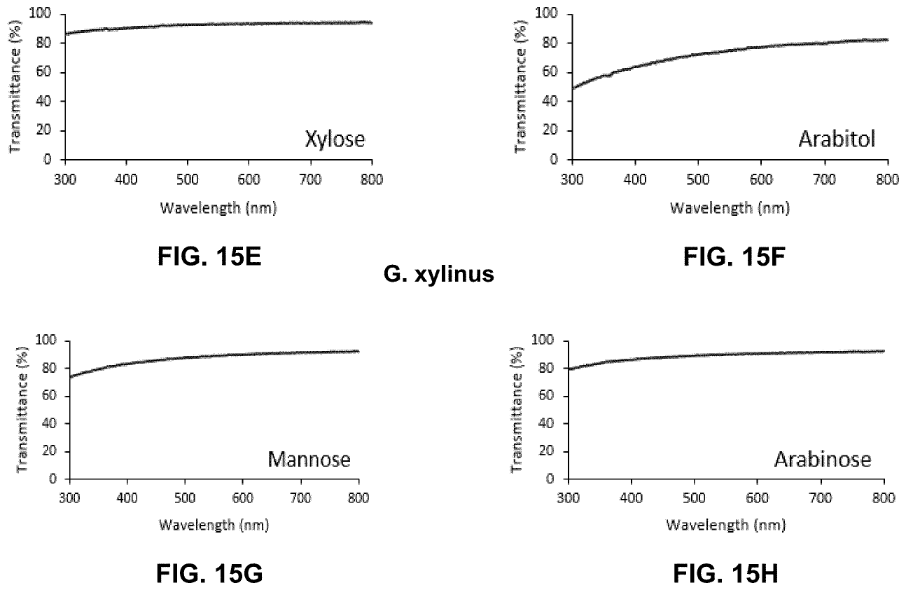

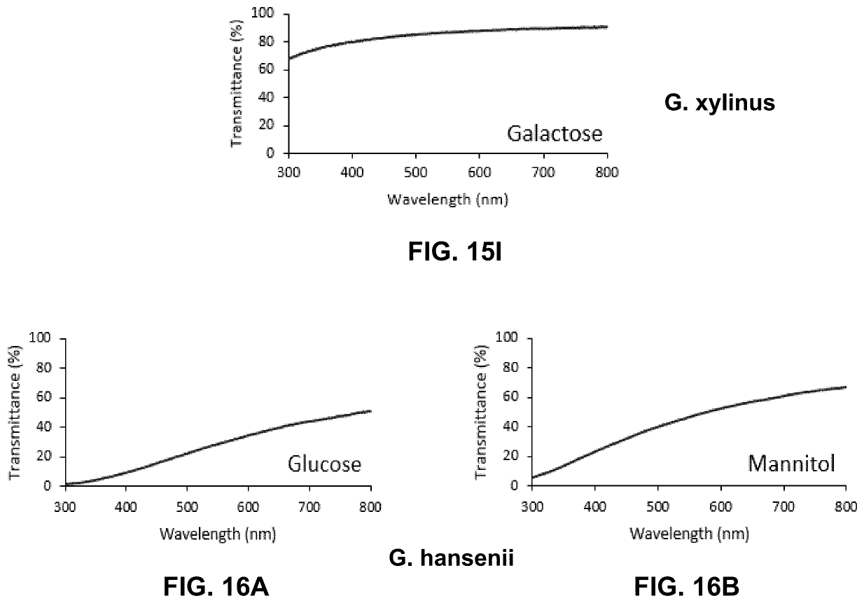

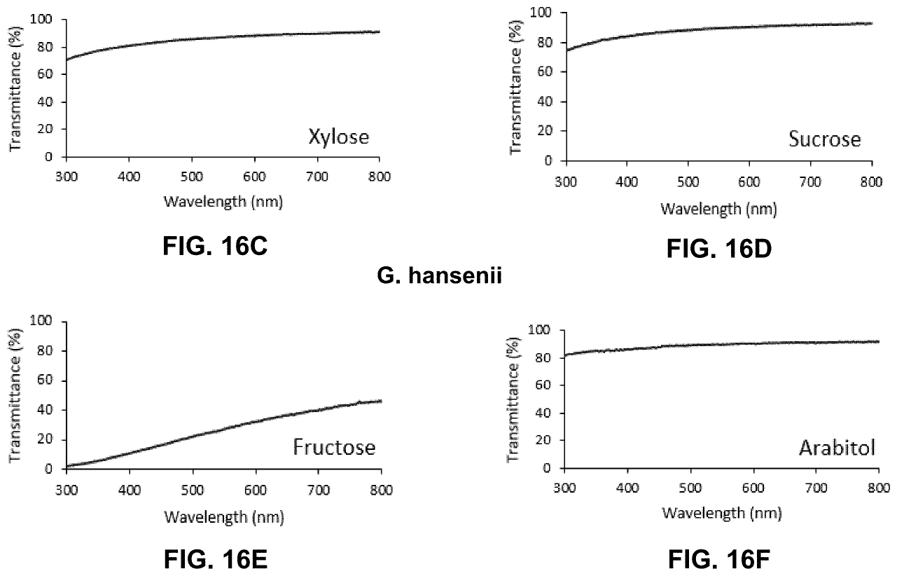

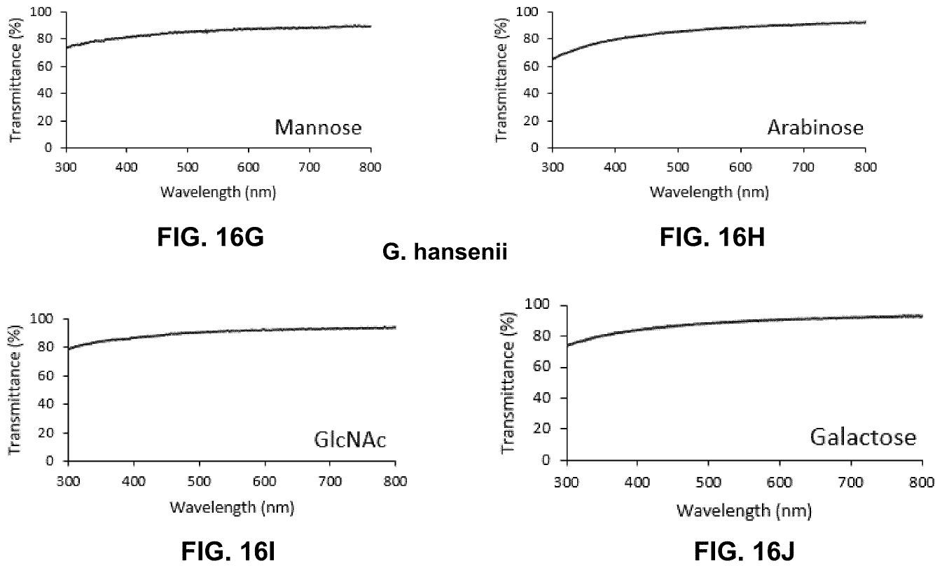

FIGS. 14A-14J provide the results for some embodiments of the disclosure grown in DS-12. FIGS. 15A-15I provide the results for some embodiments of the disclosure grown in G. xylinus. FIGS. 16A-16J provide the results for some embodiments of the disclosure grown in G. hansenii.

### Example 8: Transmittance Determination for Glucose Augmented Pellicles

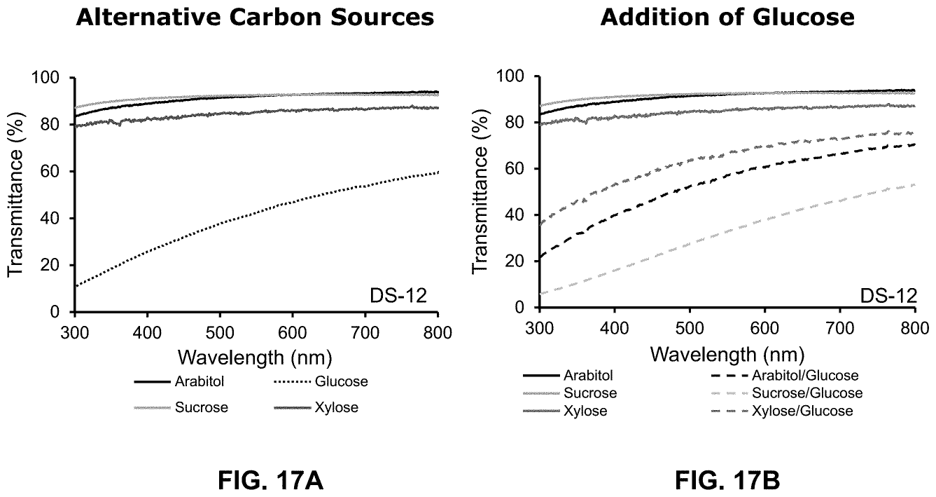

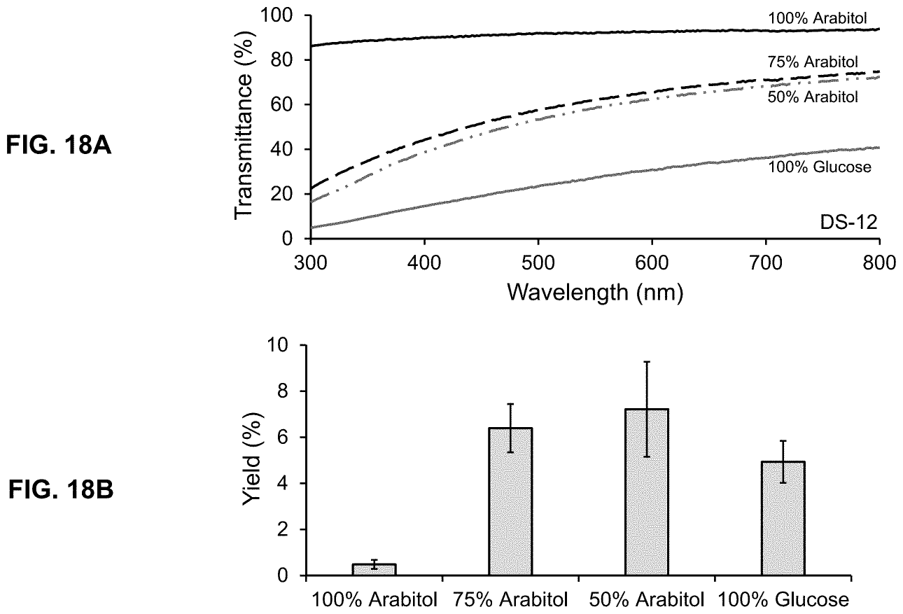

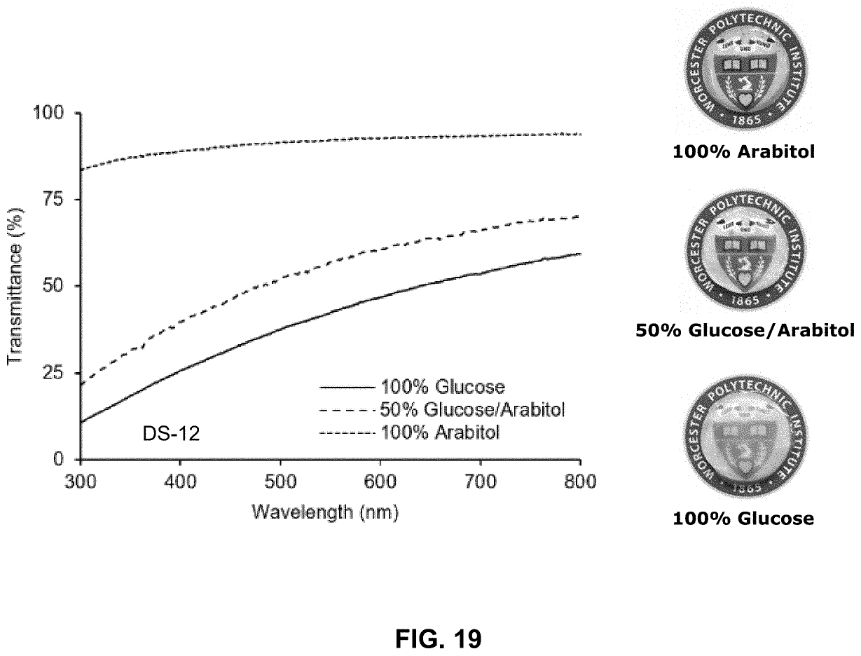

In some embodiments, glucose may be combined with an alternative carbon source. In some embodiments, increasing the percentage of glucose decreases the percent transmittance of light in a culture sample (see FIG. 17B, FIG. 18A, FIG. 19). For example, a pellicle derived from a culture receiving 100% arabitol will be more transparent than a pellicle derived from a culture receiving 75% arabitol and 25% glucose (FIG. 18A). In some embodiments, a graph of % transmittance can be determined by performing UV-Vis determinations of a light source through a sample.

FIG. 17A and FIG. 17B present graphs illustrating differences in transmittance of pellicles derived from cultures grown in alternative carbon sources (FIG. 17A) and pellicles derived from cultures grown in alternative carbon sources augmented with glucose (FIG. 17B). As shown in FIG. 17A, the pellicles derived from culture grown in alternative carbon sources arabitol, sucrose, and xylose displayed greater transmittance % versus pellicles derived from cultures grown in glucose. As shown in FIG. 17B, the pellicles derived from cultures grown in alternative carbon sources arabitol, sucrose, and xylose displayed greater transmittance % versus pellicles derived from cultures grown in alternative carbon sources arabitol, sucrose, and xylose augmented with glucose.

FIG. 18A and FIG. 18B present graphs showing transmittance percentage (FIG. 18A) and yield percentage (FIG. 18B) for pellicles produced by cultures grown in media comprising varying amounts of arabitol and glucose. As shown in FIG. 18A, pellicles produced by culture grown in media comprising 100% arabitol displayed greater transmittance % than pellicles produced by culture grown in media comprising 75% arabitol/25% glucose, 50% arabitol/50% glucose, and 100% glucose, respectively. However, as shown in FIG. 18B, pellicles produced by culture grown in media comprising 75% arabitol/25% glucose, 50% arabitol/50% glucose, and 100% glucose, respectively, displayed greater yield percentage than pellicles produced by culture grown in media comprising 100% arabitol.

Mixtures of alternative carbon sources with glucose were also tested and both DS-12 and G. xylinus showed preferential metabolism of glucose. A decrease in transparency was also observed when glucose was introduced as a carbon source, while xylose, mannose, fructose, galactose, sucrose, arabitol and arabinose produced highly transparent pellicles.

FIG. 19 presents a graph showing transmittance percentage of pellicles produced by cultures grown in media comprising 100% arabitol, 50% glucose/50% arabitol, and 100% glucose, together with photographs of the respective pellicles. As shown in FIG. 19, pellicles produced by culture grown in media comprising 100% arabitol displayed greater transmittance % than pellicles produced by culture grown in media comprising 50% arabitol/50% glucose, and 100% glucose, respectively.

### Example 9: Reduced Oxygen Conditions

FIG. 20 comprises a graph and image showing transmittance percentage of pellicles produced in ambient and low oxygen cultures of a kombucha consortium containing the DS-12 isolate and yeast using sucrose as a carbon source. As shown in FIG. 20, pellicles produced under low oxygen cultures (e.g., closed systems) displayed greater light transmittance (%) than pellicles produced under ambient oxygen cultures (e.g., open systems).

### Other Embodiments

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of preparing a transparent bacterial-derived cellulose-based material, the method comprising:
culturing cellulose-producing bacteria in a media comprising a non-glucose carbon source; and
collecting cellulose-based material produced by the cellulose-producing bacteria, wherein the cellulose-based material has a light transmission of at least 50% of light energy having a wavelength between 400 nm and 600 nm.

2. The method of claim 1, wherein the media further comprises glucose.

3. The method of claim 1, wherein the non-glucose carbon source comprises one or more of a pentose sugar, a hexose sugar, a sugar alcohol, or a disaccharide.

4. The method of claim 3, wherein the pentose sugar is selected from the group consisting of xylose and arabinose.

5. The method of claim 3, wherein the hexose sugar is selected from the group consisting of mannose, galactose, fructose, glucosamine hydrochloride, and N-acetylglucosamine.

6. The method of claim 3, wherein the sugar alcohol is selected from the group consisting of arabitol and mannitol.

7. The method of claim 3, wherein the disaccharide is sucrose.

8. The method of claim 3, wherein the non-glucose carbon source comprises one or more of mannose, galactose, xylose, mannitol, sucrose, glucosamine hydrochloride, N-acetylglucosamine, fructose, arabinose, and arabitol.

9. The method of claim 3, wherein the non-glucose carbon source comprises one or more of arabitol and xylose.

10. The method of claim 1, wherein the cellulose-producing bacteria comprises one or more bacteria from Acetobacter genus.

11. The method of claim 10, wherein the cellulose-producing bacteria comprises one or more of DS-12, Gluconacetobacter hansenii, or Gluconacetobacter xylinus bacterial species.

12. The method of claim 1, wherein the bacterial-derived cellulose-based material has a light transmission of at least 75% of light energy having a wavelength between 400 nm and 600 nm.

13. The method of claim 1, wherein oxygen tension is reduced relative to ambient.

14. The method of claim 1, wherein the non-glucose carbon source is selected to produce increased cyclic di-GMP levels in the cellulose-producing bacteria.

15. The method of claim 1, wherein the cellulose-producing bacteria is cultured without glucose.

16. A material for biomedical application comprising bacterial-derived cellulose-based material having a light transmission of at least 50% of light energy having a wavelength between 400 nm and 600 nm.

17. A kit for preparing a transparent bacterial-derived cellulose-based material comprising cellulose-producing bacteria, and
one or more non-glucose carbon sources, wherein culturing the cellulose-producing bacteria in the presence of the one or more non-glucose carbon source produces cellulose-based material having a light transmission of at least 50% of light energy having a wavelength between 400 nm and 600 nm.

18. The kit of claim 17, wherein the cellulose-producing bacteria comprises bacteria from Acetobacter genus.

19. The kit of claim 17, wherein the cellulose-producing bacteria comprises one or more of DS-12, Gluconacetobacter hansenii, or Gluconacetobacter xylinus bacterial species.

20. The kit of claim 17, wherein the non-glucose carbon source comprises one or more of mannose, galactose, xylose, mannitol, sucrose, glucosamine hydrochloride, N-acetylglucosamine, fructose, arabinose, and arabitol.
